(19) United States
(12) Patent Application Publication
(10) Pub. No.: US 2007/0219359 A1
(43) Pub. Date: Sep. 20, 2007

(54) Gene Encoding Phosphoenolpyruvate Synthase In Plant Protection

(76) Inventors: Jiliang Tang, Guangxi (CN); Yongqiang He, Guangxi (CN); Dongjie Tang, Guangxi (CN); Jiaxun Feng, Guangxi (CN); Baoshan Chen, Guangxi (CN); Guangtao Lu, Guangxi (CN); Bole Jiang, Guangxi (CN); Rongqi Xu, Guangxi (CN)

Correspondence Address: MORRISON & FOERSTER LLP, 12531 HIGH BLUFF DRIVE, SUITE 100, SAN DIEGO, CA, 92130-2040, US

(21) Appl. No.: 10/581,099

(22) PCT Filed: Dec. 1, 2004
(86) PCT No.: PCT/CN04/01386

§ 371 Date: Oct. 10, 2006

(30) Foreign Application Priority Data
Dec. 1, 2003 (CN) 200310116875.X

(51) Int. Cl. C07H 21/04; A01N 43/00

(52) U.S. Cl. 536/23.1; 504/191

(57) ABSTRACT

The present invention discloses a pathogenic gene derived from Xanthomonas campestris, a gene encoding phosphoenolpyruvate synthase. The gene encoding phosphoenolpyruvate synthase of this invention has one of the following nucleotide sequence: 1. a nucleotide sequence of SEQ ID NO:1; 2. a DNA sequence which has more than 80% homology with the nucleotide sequence of SEQ ID NO:1, and encodes a protein which has same function as phosphoenolpyruvate synthase encoded by SEQ ID NO:1. The Open Reading Frame of the DNA of SEQ ID NO:1 is from nucleotide 201 to 2576 in its 5′ end. It consists of 2379 nucleotides, the initiation codon TTG of this gene is from nucleotides 201 to 203 in its 5′ end, and the termination codon TGA of this gene is from nucleotides 2577 to 2579 in its 5′ end.

### FIELD OF THE INVENTION

The present invention relates to a novel pathogenicity gene of plant bacterial pathogen. This gene encodes phosphoenolpyruvate synthase (ppsA) which is the key enzyme in gluconeogenesis. The gene and its product can be used as a novel target for plant disease control.

### DESCRIPTION OF THE PRIOR ART

Plant disease is one of the most destructive factors lead to poor quality and low yield of crops. As the pathogen resistance to antibiotics and pesticides increases, the amount of pesticides is increasingly used in agricultural practices, which result in heavily environmental pollution and pesticide residue in crop production, and also raise agricultural costs. The environment friendly strategies and chemicals are pressingly needed to be developed. The key to the development of novel chemicals and measurements in disease control is to understand the genetic mechanism of pathogenesis of plant pathogens, and genomics study provides novel approaches to explore the functions of the genes and the mechanisms of the interactions between plants and the pathogens on molecular level. Along with the development of DNA sequencing technology, the hot spots of the genomics are shifted from structural genomics, the main tasks of which are DNA sequencing, into the functional genomics where the identification of the functions of ORF and gene is becoming increasingly dominant. The high-throughput and large-scale works on the identification of gene function has laid a fundamental basis for newly developed biotechnology based industry. To date, pathogenic genes which have been identified include hypersensitive reaction and pathogenicity gene (hrp) (He 1998), avirulence genes (avr) (Bonas 1999), regulatory pathogenicity factors (rpf) (Barber 1997), gene involved in biosynthesis of exopolysaccharides (Tang, et al. (1991); Dow, et al. (2000a)), gene involved in biosynthesis of lipopolysaccharides (Dow, et al. (2000b)), etc.

Determining of the function of proteins predicted in the genome sequence is the major goal for the post-genome era. One of the direct ways for functional assignments is disrupting the gene and examining the phenotype of the mutant. The genome-wide mutagenesis and mutant collection would provide important resources for the researches in bio-technology and life science on the genome level.

Xanthomonas campestris pathovar campestris (Xcc), a yellow pigmented γ-proteobacterium, is the causal agent of black-rot disease of crucifers, one of the most destructive diseases of cruciferous plant worldwide (Hayward, 1993). The present invention was based on the construction of mutant library of Xcc 8004 and genome-wide identification of novel pathogenicity genes and intended to provide the pathogenicity genes and their products to be used as a novel target for plant disease control.

### DETAILED DESCRIPTION OF THE INVENTION

It is an object of the present invention to provide a novel gene related to Xanthomonas campestris associated diseases.

The present invention provided the objective gene, phosphoenolpyruvate synthase gene, which has one of the following nucleotide sequences:

1. the nucleotide sequences of SEQ ID NO:1; and

2. a DNA sequence which has more than 80% homology with SEQ ID NO:1 and encodes a protein having the same function as the phosphoenolpyruvate synthase encoded by the sequence of SEQ ID NO:1.

The present invention provided a phosphoenolpyruvate synthase gene. Preferably, the gene has the DNA sequence of SEQ ID NO:1.

Plasmid pXC1950 harboring the phosphoenolpyruvate synthase gene was deposited in China General Microbiological Culture Collection Center on 27^{th} Nov. 2003, with the accession number of CGMCC No. 1054, the identification reference of JM109/pXC1950, and the taxonomic designation of Escherichia coli.

The DNA sequence of SEQ ID NO:1 is part of the genomic DNA of Xcc 8004 strain. SEQ ID NO:1 consists of 2629 nts and contains the entire phosphoenolpyruvate synthase gene. The open reading frame (ORF) of the gene starts from 201 nt from 5′-end to 2576 nt; the start codon TTG of the gene starts from 201 nt from 5′-end to 203 nt; the stop codon TGA of the gene starts from 2577 nt from 5′-end to 2579 nt, and the promoter region of the gene starts from 120 nt from 5′-end to 170 nt.

In the present invention, DNA sequences with sequence similarity of greater than 85%, 90%, 95%, 98% or 99% of SEQ ID NO:1 and encoding proteins having the same function of the phosphoenolpyruvate synthase encoded by SEQ ID NO:1, are preferred.

The amino acid sequence of SEQ ID NO:2 represents the deduced amino acid sequence of the phosphoenolpyruvate synthase gene, which consists of 792 amino acid residuals, with a molecular weight of 86.2 KD and an isoelectric point of 5.12.

The deduced phosphoenolpyruvate synthase consists of 792 amino acid residuals, and contains the pyruvate binding domain and the PEP-utilizing enzyme mobile domain.

The expression vectors containing the DNA sequences mentioned above are also intended to be included in the present invention.

The use of XC1950 gene: Phosphoenolpyruvate synthase is the key enzyme in gluconeogenesis in bacterial pathogen. The inventors have discovered that gluconeogenesis is very important to the bacterial adaptation to adverse effects. Previous study has shown that the disruption of gluconeogenic pathway resulted in significant reductions in Xcc 8004 virulence. Inventors also have discovered the mutation of the phosphoenolpyruvate synthase will block the gluconeogenic pathway. That is to say, one can control the toxicity of bacteria if he/she can control the activity of phosphoenolpyruvate synthase gene or its product. Therefore, phosphoenolpyruvate synthase gene and its product can be used as targets in medicament treatment and plant disease control.

### DESCRIPTION OF THE DRAWINGS

FIG. 1 shows the electrophoresis result of digested XC1950 gene clone.

Lane 1: λ/HindIII DNA markers (DNA markers, from the largest to the smallest: 23.1 kb, 9.4 kb, 6.6 kb, 2.4 kb, and 2.0 kb). Lane 2: The fragments of XC1950 gene. Lane 3: The constructed plasmid pXC1950 digested with BamHI+HindIII.

FIG. 2 shows the electrophoresis result of the PCR result of XC1950 deletion mutant.

Lane 1: 100 bp DNA marker (DNA markers, from the largest to the smallest: 3 kb, 2 kb, 1.5 kb, 1.2 kb, 1 kb, 0.9 kb, 0.8 kb, and 0.7 kb). Lane 2: Total DNA from wild type Xcc 8004 strain as the templates. Lane 3-6: Total DNA from XC1950 deletion mutants as the templates.

FIG. 3 shows the growth pattern of XC1950 mutants on medium which has pyruvate as its sole carbon source.

Colonies 1-4: The Tn5 insertion mutants of XC1950 gene. Colonies 5-6: The deletion mutants of XC1950 gene. Colonies 7-8: The complimentary strains of deletion mutants of XC1950 gene. Colonies 9-10: Wild type Xcc 8004 strain.

FIG. 4 shows the result of pathogenicity test of XC1950 deletion mutants.

A. Wild type Xcc 8004 strain. B. The deletion mutants of XC1950 gene. C. Water (used as negative control)

The following examples are offered to illustrate but not to limit the invention.

### EXAMPLES

### Materials Used in Examples

Escherichia coli JM109 strain and plasmid pGEM-3Zf(+) were purchased from Promega, Shanghai. Restriction enzymes and modification enzymes were purchased from Promega, Stratagene, and QIAGEN companies.

The host plant used in the example is Chinese radish (Raphanus sativus L. var. radiculus Pers.), which was purchased from Sichuan Seed Company, China.

Wild type Xcc 8004 (See, for example, Tang J L et al. 1990. Cloning of genes involved in negative regulation of production of extracellular enzymes and polysaccharide of Xanthomonas campestris pathovar campestris. Mol Gen Genet. 222:157-160.); The Cosmid pLAFR1 and pLAFR3 (See, for example, Liu Y N et al. 1990. A multipurpose broad host range cloning vector and its use to characterize an extracellular protease gene of Xanthomonas campestris pathovar campestris. Mol Gen Genet. 220:433-440.); Cosmid pPH1JI (See, for example, Hirsch P R et al. 1984. A physical map of pPH1JI and pJB4JI. Plasmid. 12:139-141.); Transposon Tn5gusA5 (See, for example, Zha D et al. 1998. Cloning of DNA sequences involved in exopolysaccharide synthesis of Xanthomonas campestris pv. campestris. Wei Sheng Wu Xue Bao. 38:251-255.).

Nutrient yeast extracts and glycerol (NYG) medium, containing 5.0 g/L polypeptone, 5.0 g/L yeast extracts, and 20.0 g/L glycerol, pH7.0.

Primers for gene XC1950 amplification:

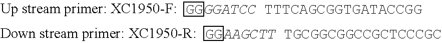

The italicized letters indicate the additional restriction sites (BamHI, Hind III); the squared letters indicate the protection nucleotides for restriction sites.

### Example 1

### Construction of Mutant Library of Xcc

Cosmid pLAFR1 which can replicate in both E. coli cell and Xcc cell was used as the vector (pLAFR1::Tn5gusA5) to introduce the transposon Tn5gusA5 into the wild type Xcc 8004 strain. The bacterial strain thus created was named as Xcc 8004/pLAFR1::Tn5gusA5. Incompatible plasmid pPH1JI was then introduced into Xcc 8004/pLAFR1::Tn5gusA5 to drive out the pLAFR1::Tn5gusA5. Insertional mutants Xcc:: Tn5gusA5 was screened on NYGA medium containing antibiotics (Kanamycin). The obtained DNA sequences were performed sequence alignment to the whole genome sequence of Xcc 8004 and the insertional site of each Tn5gusA5 in the chromosome of the mutant was determined by TAIL-PCR (Thermal Asymetric Interlaced-PCR) technique (Liu et al. 1995). Totally, 420 mutants with defects associated with pathogenicity, extracellular enzymes and polysaccharides were colleted, 51 genes involved. Of the 51 genes, five were newly identified pathogenicity genes. The present invention relates only to XC1950, one of the five genes.

The deduced product of XC1950 was predicted as phosphoenolpyruvate synthase and the BLASTP (a web-based bioinformatics tools developed by National Center for Biotechnology Information of USA, available at http://www.ncbi.nlm.nih.gov/BLAST/) result showed that XC1950 contains phosphoenolpyruvate synthase/pyruvate phosphate dikinase domain at its N-terminal and PEP-utilizing enzyme domain at its C-terminal, indicating XC1950 is phosphoenolpyruvate synthase gene and is involved in gluconeogenesis.

### Example 2

### The Cloning of XC1950 Gene (Phosphoenolpyruvate Synthase Gene)

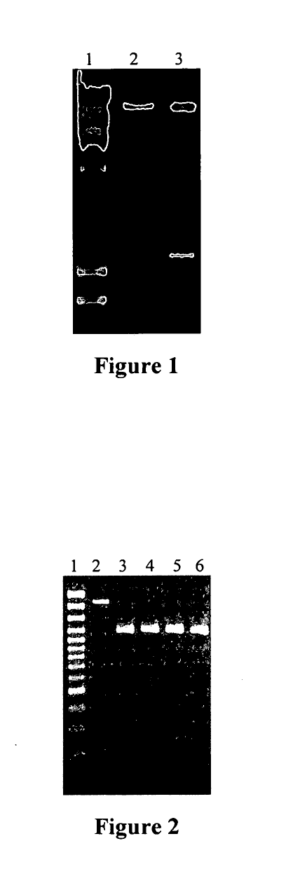

Primers (XC1950-F and XC1950-R) were designed according to the DNA sequence of XC1950 gene. The intact DNA sequence of XC1950 gene was amplified by PCR, using XC1950-F and XC1950-R as primers and the total genomic DNA of wild type Xcc 8004 as template (FIG. 1). The PCR product was cloned into pLAFR3, and the recombined plasmid was designated as pXC1950. The fragment of XC1950 gene was subcloned from pXC1950 into pGEM3Zf(+) for long-term storage.

### Example 3

### The Construction and Confirmation of the Deletion Mutant of XC1950 Gene

The Kanamycin gene (Kan) was amplified by PCR and cloned into vector pGEM3Zf(+). The up-stream and down-stream flanking sequences of XC1950, designated as 1950L and 1950R, were obtained by PCR. 1950L and 1950R were recombined to both ends of the cloned Kan fragment in pGEM3Zf(+), producing recombinant plasmid pGK1950 in which the 1950L, Kan fragment, and 1950R were tandemly ligated. Then, the fragment 1950L-Kan fragment-1950R was subcloned into pLAFR3, producing pLGK1950. The pLGK1950 was introduced into wild type Xcc 8004 strain and the Kan fragment was replaced by XC1950 gene via homologous cross-over at both 1950L and 1950R positions. After the introduction of incompatible plasmid pPH1JI by bi-parental mating, the conjugants were screen through culturing on NYGA medium containing antibiotics. The positive conjugants were confirmed by specific PCR. As shown in FIG. 2, the deletion mutants and the wild type Xcc 8004 produced significantly different product band patterns.

### Example 4

### The Growth Pattern of the Mutants of XC1950 Gene on Culture Medium with Pyruvate as the Sole Carbon Source

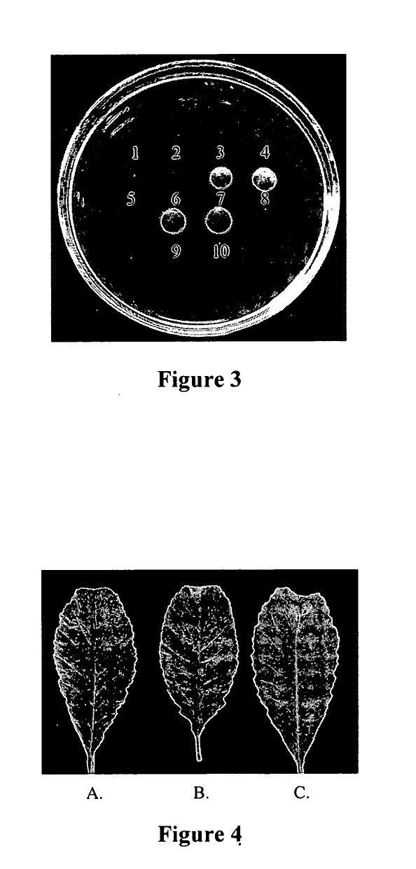

On solid medium with pyruvate as the sole carbon source (2.0 g/L (NH₄)₂SO₄, 1 g/L Pyruvate, 4.0 g/L K₂HPO₄, 6.0 g/L KH₂PO₄, and 0.2 g/L MgSO₄.7H₂ O, 15 g/L Agar, pH7.0), wild type Xcc 8004 strain, mutants, and the complimentary strain (mutant carrying the pXC1950) were inoculated by tip-dipping using autoclaved toothpicks and cultured under 28° C. for 72 hours. The results showed that the mutants could not grow normally, but the wild type strain and the complimentary strain grew normally on the culture medium (FIG. 3), demonstrating that the mutation in XC1950 gene caused Xcc defective in pyruvate utilization.

### Example 5

### The Pathogenicity Test of XC1950 Gene Mutant

The host plant used in the example is Chinese radish (Raphanus sativus L. var. radiculus Pers.) and the leaf-clipping was used as inoculating method. The deletion mutants and the wild type strain were cultured in suspensions at 28° C. for 15-18 hours, and were diluted to OD600=0.2 before use. The mutants and the strains were inoculated into the radish leaves by cutting along the mid-vein of the leaf from a distance of 1-2 cm from the leaf tip with clippers which have been dipped in the suspensions for 5 seconds. The inoculated plants were kept in the warm and moisture greenroom at 25-30° C. for 7 days. The wild type strain was used as positive control, and the water as the negative control. The result of plant test showed that the pathogenicity of mutants decreased significantly compared with the wild type and the complimentary strain, which indicated that the XC1950 gene was directly involved in the pathogenicity of Xcc.

### REFERENCES

Barber C E et al. 1997. A novel regulatory system required for pathogenicity of Xanthomonas campestris is mediated by a small diffusible signal molecule. Mol. Microbiol 24:555-566
Bonas U et al. 1999. Gene-for-gene interactions: bacterial avirulence proteins specify plant disease resistance. Curr. Opinion in Microbiology 2:94˜98
Dow M et al. 2000a. Xylella genomics and bacterial pathogenicity to plants. Yeast 17:263˜271.
Dow M et al. 2000b. The induction and modulation of plant defense resistanses by bacterial lipopolysaccharides. Annu Rev Phytopathol 38:241-261.
Hayward A C. 1993. The hosts of Xanthomonas. In: Swings G, Civerolo E L, editors. Xanthomonas. London: Chapman and Hall, pp. 1-119.
He SY. 1998. Type III protein secretion systems in plant and animal pathogenic bacteria. Annu. Rev. Phytopathol. 36:363˜392
Hirsch P R et al. 1984. A physical map of pPH1JI and pJB4JI. Plasmid. 12:139-141.
Liu Y G et al. 1995 Efficient isolation and mapping of Arabidopsis thaliana T-DNA insert junctions by thermal asymmetric interlaced PCR. Plant J. 8:457-63.
Liu Y N et al. 1990. A multipurpose broad host range cloning vector and its use to characterise an extracellular protease gene of Xanthomonas campestris pathovar campestris. Mol Gen Genet. 220:433-440.
Tang J L et al. 1991. Genetic and molecular analysis of a cluster of rpf genes involved in positive regulation of synthesis of extracellular enzymes and polysaccharide in Xanthomonas campestris pathovar campestris. Mol Gen Genet 226:409-417.
Zha D et al. 1996. Mutagenesis of Xanthomonas campestris pv. campestris with transposon Tn5gusA5 and isolation of mutants impaired with pathogenicity. Journal of Guangxi Agricultural and Biological Science. 15:23-29.
Zha D et al. 1998. Cloning of DNA sequences involved in exopolysaccharide synthesis of Xanthomonas campestris pv. campestris. Wei Sheng Wu Xue Bao. 38:251-255.

1-5. (canceled)

6. Gene XC1950, which encodes phosphoenolpyruvate synthase and has one of the following sequences:
the nucleotide sequence of SEQ ID NO:1; and
a DNA sequence which has more than 80% homology with SEQ ID NO:1 and encodes a protein having the same function as the phosphoenolpyruvate synthase encoded by the sequence of SEQ ID NO:1.

7. The gene of claim 6, wherein the gene has the DNA sequence of SEQ ID NO:1.

8. The gene of claim 6 wherein the Open Reading Frame of the gene is from nucleotide 201 to 2576 from its 5′ end.

9. The gene of claim 7 wherein the Open Reading Frame of the gene is from nucleotide 201 to 2576 from its 5′ end.

10. Use of the gene of claim 6 in preventing and treating plant diseases.

11. Use of claim 10, wherein the gene can be used as a target for medicament treatment.


SEQUENCE LISTING (4 sequences)

SEQ ID NO: 1 - DNA, 2629 bp, Xanthomonas campestris pathovar campestris
tttcagcggtgataccggttccatcggacacgtaaaacaccggccggattgttgacatcgactccacctctttgaactagtgtccaaaacttacgggttcatgcttgtgcaggctccgctgtgcactgcatcatagcggcttcttcctacggtcgcggccattcagcccgctcgggcgatggccatacggagcatcgcgcttgaacgagaatatcctgtggttgcatgagctacgcctggtcgatctggcccgcgtaggcggtaaaaattcctcgctcggcgagatgatcggcaacctggccgggttgggcgtttcggttcccggtggatatgcgaccactgccgaagcattcaaggacttcatcgcgcacaacgatctgtcaaagcgcattttcgacaagctggagacgctggacgttgaagacgtcaccgcgctcacggtcgccggcaaggagatccgcggctgggtgatcgacgccccgctgcagccggagctggaccgcgacatccgcagcgcctacgaaaaactctgcgccgagaacggcggcggcgaagtggccgtggctgtgcgttcgtcggcaaccgccgaagacctgcccgatgcctcgttcgccggccagcaggaaaccttcctcaatgtgaccggcgccgacgacgtggtgcacaaggtcaaggaagtattcgccagcctctacaacgaccgcgcgattgcctaccgcgtgcaccacggcttcaagcacgaagatgtgttcctgtcggccggcgtgcagttgatggtgcgctccggcgtgggttcgtccggcgtgttgttcaccctggacaccgagtccggcttccgcgacgtggtgttcgtcacctccagcttcggcctgggcgaaatggtcgtgcaaggcgcggtcaatccggacgagttctacgtctacaagcccacgctcactgcgggcaagccggcaatcctgcgccgctcgctcggcagcaaggcaatccgcatggtgtattcggatgtgcccggtgaacgcgtgcgcatcgaagacacgccggtggagttgcgcaacactttctcgatcagcgacgaagatgtgcaggagctctccaagcaggcgctggtgatcgaaaagcattacggccgcccgatggatatcgagtgggccaaggacggcgtgagcggcaagctgttcatcgtgcaggcgcgcccggagacggtgaagtcgcgcagccatgccacccagatcgagcgtttctcgctggaagccaaggacgccaagatcctggtcgaaggccgtgcggttggcgccaagatcggcagcggcgtggcacgcgtggtgcgctcgctggaagacatgaatcgcgtgcaggccggcgacgtgctgattgccgacatgaccgaccccgattgggagccggtgatgaagcgtgcctcggccatcgtcaccaaccgcggtggccgcacctgccacgcggcgatcatcgcgcgcgaactgggcgtgccggcggtggtgggttcgggcaatgcgaccgacgtcatcagcgacggccaggaagtcaccgtgagctgcgccgagggcgacaccggcttcatctatgaaggcttgctgccgttcgagcgcaccaccaccgacctgggcaacatgccgcctgccccgctcaagatcatgatgaacgtggccaacccggagcgcgcattcgacttcggccagctgcccaacgccggtatcggcttggcgcgtctggagatgatcatcgccgcgcacatcggcatccatcccaacgcactgctggaatacgacaagcaggacgccgacgtccgcaagaagatcgacgccaagattgccggctacggcgacccggtgagcttctacatcaaccgcctggccgaaggcatcgcgaccctgaccgcgtcggtggcgccgaacacggtgatcgtgcggttgtcggacttcaagtccaacgaatacgccaacctgatcggtggctcgcgttacgagccgcacgaagagaacccgatgatcggcttccgcggcgccagccgttatgtcgatccgtccttcaccaaggcgttctcgctggagtgcaaggcggtgttgaaggtgcgcaacgagatgggcctggacaacctctgggtcatgattccgttcgtgcgcacgctggaggaaggccgcaaggtgatcgaggtgttggagcagaacgggctcaaacaaggcgagaacgggctgaagatcatcatgatgtgcgagctgccgtccaatgcgctgctggccgatgagttcctggagatcttcgacggcttctcgatcggctccaacgacctgacccagctcaccctgggcctggaccgcgattcctcgatcgtggcgcacctgttcgacgagcggaacccggcggtgaaaaagctgctgtcgatggcgatcaagtcggcgcgggccaagggcaagtacgtgggcatctgcggccaggggccgtcggatcacccggaactggccgagtggttgatgcaggaaggcatcgagtcggtgtcgctgaatcctgacaccgtggtcgatacctggctgcgcctggccaagctcaagagcgagggctgatgggaatggtgggtggagtgttggcggcggctgcgggagcggccgccgca

SEQ ID NO: 2 - protein, 792 aa, Xanthomonas campestris pathovar campestris
LNENILWLHELRLVDLARVGGKNSSLGEMIGNLAGLGVSVPGGYATTAEAFKDFIAHNDLSKRIFDKLETLDVEDVTALTVAGKEIRGWVIDAPLQPELDRDIRSAYEKLCAENGGGEVAVAVRSSATAEDLPDASFAGQQETFLNVTGADDVVHKVKEVFASLYNDRAIAYRVHHGFKHEDVFLSAGVQLMVRSGVGSSGVLFTLDTESGFRDVVFVTSSFGLGEMVVQGAVNPDEFYVYKPTLTAGKPAILRRSLGSKAIRMVYSDVPGERVRIEDTPVELRNTFSISDEDVQELSKQALVIEKHYGRPMDIEWAKDGVSGKLFIVQARPETVKSRSHATQIERFSLEAKDAKILVEGRAVGAKIGSGVARVVRSLEDMNRVQAGDVLIADMTDPDWEPVMKRASAIVTNRGGRTCHAAIIARELGVPAVVGSGNATDVISDGQEVTVSCAEGDTGFIYEGLLPFERTTTDLGNMPPAPLKIMMNVANPERAFDFGQLPNAGIGLARLEMIIAAHIGIHPNALLEYDKQDADVRKKIDAKIAGYGDPVSFYINRLAEGIATLTASVAPNTVIVRLSDFKSNEYANLIGGSRYEPHEENPMIGFRGASRYVDPSFTKAFSLECKAVLKVRNEMGLDNLWVMIPFVRTLEEGRKVIEVLEQNGLKQGENGLKIIMMCELPSNALLADEFLEIFDGFSIGSNDLTQLTLGLDRDSSIVAHLFDERNPAVKKLLSMAIKSARAKGKYVGICGQGPSDHPELAEWLMQEGIESVSLNPDTVVDTWLRLAKLKSEG

SEQ ID NO: 3 - DNA, 26 bp, Artificial Sequence
  Up stream primer
ggggatcctttcagcggtgataccgg

SEQ ID NO: 4 - DNA, 26 bp, Artificial Sequence
  Down stream primer
ggaagctttgcggcggccgctcccgc